(19) United States
(12) Patent Application Publication
(10) Pub. No.: US 2021/0134178 A1
(43) Pub. Date: May 6, 2021

(54) TWELVE-PHASE HOLISTIC HEALTH TRAINING SYSTEM AND METHOD

(71) Applicant: Robert Killen, Suffolk, VA (US)

(72) Inventor: Robert Killen, Suffolk, VA (US)

(21) Appl. No.: 16/672,452

(22) Filed: Nov. 2, 2019

(51) Int. Cl. G09B 19/00; G16H 20/30

(52) U.S. Cl. CPC G09B 19/00; G09B 19/0092; G16H 20/30

(57) ABSTRACT

A twelve-phase holistic health training system and method is disclosed. The system and method may comprise, at least, twelve primary phases and a thirteenth, overarching, phase to assist a person in achieving holistic wellness. The primary phases may include, for example, perspective, environment, appreciation, career, education, balance, exercise, nutrition, evolution, faith, income, and time, and the thirteenth phase may comprise security. The training in and application of the various phases are central to the achievement of overall wellness.

### TECHNICAL FIELD OF THE INVENTION

The present invention relates in general to holistic training, and, more specifically, to a twelve-phase holistic health training system and method.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

A life coach helps clients develop, meet, and exceed goals in their personal and professional lives. Life coaching is a professional, supportive, co-creative relationship where both coach and client collaborate to form an action plan to reach new personal and professional goals. Many famous celebrities, business leaders, politicians, and professional athletes have utilized life coaches to help them reach their full potential. Bill Gates, Oprah Winfrey, Leonardo DiCaprio, Bill Clinton, Barrack Obama, Serena Williams, Andre Agassi, Chuck Liddell and even the rock band, Metallica, have all worked with coaches. Bill Gates has gone on record to say that “everyone needs a coach.”

The key differences between therapy and life coaching are many. Therapy focuses more on overcoming the past and can be more clinical, medical, and boundaried. It focuses on improving mental health and dysfunction, provides insight and healing of traumatic experiences, provides a safe place to share feelings, tends to be more time limited, uses labels for dysfunctional behavior, and the results can be felt mostly internally.

Life coaching, on the other hand, focuses more on the present and future and can be more warm, personal, and holistic. Such coaching focuses on positive action, growth, unlocking potential, and achieving goals. It also provides insight, accountability, motivation, inspiration, and can decrease stress and improve self-esteem. Life coaching challenges a person's beliefs and perspectives and tends to have longer sessions including more regular contact. The removal of labels allows a person to reclaim their core identity and enable further growth, both internally and externally.

Holistic Wellness is a level of self-care that encompasses the whole person—body, mind, spirit, and emotions—and is considered ideal for those seeking to achieve the optimal level of health. It is important to address all the key areas to live a life of balance, purpose, peace, health, and happiness. These key areas may be called the Peace Benefits.

A dodecagon is a geometric figure with twelve sides. The Peace Dodecagon represents the twelve areas that comprise all the dimensions of wellness—the Peace Benefits. Learning how to understand and apply the first 12 Peace Benefits principles creates the ultimate level of peace and security, which is the thirteenth and final Peace Benefit.

There is a need in the art for a twelve-phase holistic health training system and method that is employed by a trained holistic professional for more effective training. It is to these ends that the present invention has been developed.

### BRIEF SUMMARY OF THE INVENTION

To minimize the limitations in the prior art, and to minimize other limitations that will be apparent upon reading and understanding the present specification, the present invention describes a twelve-phase holistic health training system and method.

It is an objective of the present invention to provide a twelve-phase holistic health training system and method that may comprise a holistic training system.

It is another objective of the present invention to provide a twelve-phase holistic health training system and method that may comprise a holistic training method.

It is another objective of the present invention to provide a twelve-phase holistic health training system and method that may comprise a twelve-phase system.

It is another objective of the present invention to provide a twelve-phase holistic health training system and method that may comprise a perspective phase.

It is another objective of the present invention to provide a twelve-phase holistic health training system and method that may comprise an environment phase.

It is another objective of the present invention to provide a twelve-phase holistic health training system and method that may comprise an appreciation phase.

It is another objective of the present invention to provide a twelve-phase holistic health training system and method that may comprise a career phase.

It is another objective of the present invention to provide a twelve-phase holistic health training system and method that may comprise an education phase.

It is another objective of the present invention to provide a twelve-phase holistic health training system and method that may comprise a balance phase.

It is another objective of the present invention to provide a twelve-phase holistic health training system and method that may comprise an exercise phase.

It is another objective of the present invention to provide a twelve-phase holistic health training system and method that may comprise a nutrition phase.

It is another objective of the present invention to provide a twelve-phase holistic health training system and method that may comprise an evolve phase.

It is another objective of the present invention to provide a twelve-phase holistic health training system and method that may comprise a faith phase.

It is another objective of the present invention to provide a twelve-phase holistic health training system and method that may comprise an income phase.

It is another objective of the present invention to provide a twelve-phase holistic health training system and method that may comprise a time phase.

It is another objective of the present invention to provide a twelve-phase holistic health training system and method that may comprise a security phase.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 schematically illustrates a twelve-phase holistic health training system and method, as contemplated by the present disclosure; and

FIG. 2 schematically presents a computing system configured to carry out and actualize methods and tasks described herein, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

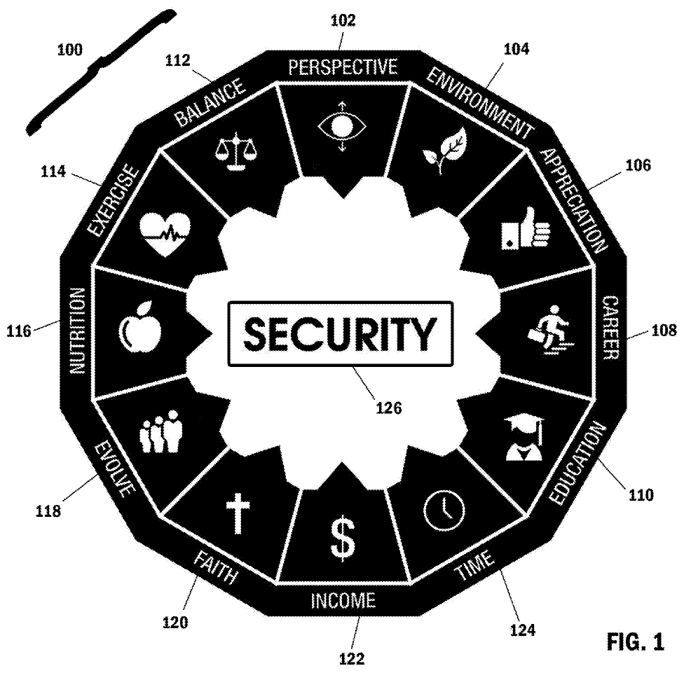

The illustration of FIG. 1 illustrates a twelve-phase holistic health training system and method, as contemplated by the present disclosure. The system and method may comprise, at least, twelve primary phases and a thirteenth, overarching, phase to assist a person in achieving holistic wellness. The primary phases may include, for example, perspective, environment, appreciation, career, education, balance, exercise, nutrition, evolution, faith, income, and time, and the thirteenth phase may comprise security. The training in and application of the various phases are central to the achievement of overall wellness.

The twelve-phase holistic health training system and method may be represented by a dodecagon 100, which may comprise the twelve primary phases and the thirteenth overarching phase.

The perspective phase 102 represents a beginning in the quest for peace, health, and happiness. Adopting the right mindset is the single most important phase because how a user views itself, its circumstances, and others is the foundation that ultimately determines the user's level of success in mastering all other the phases.

The environment phase 104 requires that the user place itself in the ideal surroundings with people who enable and support the user's personal growth and improvement.

The appreciation phase 106 allows the user to identify the three different types of appreciation in its life to achieve unprecedented levels of personal growth and peace.

The career phase 108 requires that the user learn to master the 5 keys to successfully choose the right career for it. Life is too short not to be happy and fulfilled in work.

The education phase 110 teaches the user to become a life-long learner as a necessity for life to continue being exciting, interesting, productive, and fulfilling.

The balance phase 112 is the single most often overlooked, misplaced, and forgotten phase. Until the user learns how, what, and when to say “no,” and what needs to be reduced or let go, the user will never experience a life of ultimate peace.

The exercise phase 114 teaches the user to increase exercise to effective levels. According to the CDC, over 75% of Americans do not get the recommended amount of exercise. The people who do exercise regularly look and feel better, suffer from less sickness and disease, are psychologically healthier, have greater balance in their life, are happier, and make more money.

The nutrition phase 116 teaches that the second most important thing after what you feed your mind is what you feed your body. It is impossible to look and feel the absolute best until a user learns how to fuel its body with quality foods in the right quantities.

The evolve phase 118 epitomizes the essence of the various phases, namely to help initiate the changes necessary to gradually develop into a more complex and complete person.

The faith phase 120 is the one phase that, on its own, produces the greatest level of peace. Faith allows the user to endure, persist, and continue on in the pursuit of goals during the most difficult times in life. Faith in God allows the user to tap into the single greatest resource in the universe, the One who created it. God knows the user better than the user knows itself, and He is the author and creator of any gifts, talents, skills, or abilities the user has now, and will have in the future. There is no substitute for learning how to exercise Godly wisdom and discernment in all decision making throughout life.

The income phase 122 is an ongoing lifetime requirement. Money should be viewed as a tool. When used properly it can provide opportunity, education, training, support, and assistance to the user, its family, and countless other businesses and organizations that serve others around the world. For all the good many can do when used properly, it is also responsible for much of the world's havoc, destruction, corruption, violence, injustice, and death.

The time phase 124 is the single greatest resource that all persons have the same amount of, no matter their position in life. How the user chooses to spend its time will determine the outcome for its life because anything done will require time, and once it is gone it cannot be gotten back. One can tell everything they need to know about a person by how they spend their time and money.

The security phase 126 comprises the sum total of learning how to master the other twelve phases. This is the best method to obtaining the ultimate level of peace, health, and happiness in the user's life.

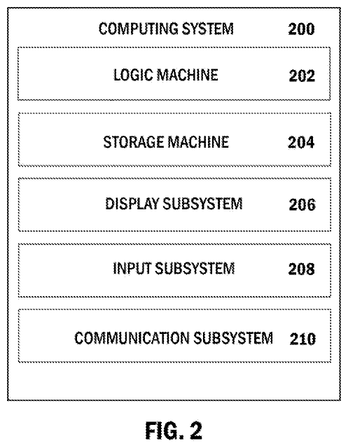

The illustration of FIG. 2 schematically presents a computing system that may represent an embodiment of the present invention. In some embodiments the method is executed on a computing system such as computing system 200. For example, storage machine 204 may hold instructions executable by logic machine 202 to provide the method to users.

Display subsystem 206 may display the various elements of the method to participants. For example, display subsystem 206, storage machine 204, and logic machine 202 may be integrated such that the method may be executed while being displayed on a display screen. The input subsystem 208 may receive user input from participants to indicate the various choices or user inputs described above.

The described method may be executed, provided, or implemented to a user on one or more computing devices via a computer-program product such as via an application programming interface (API). Computing system 200 may be any appropriate computing device such as a personal computer, tablet computing device, gaming device or console, mobile computing device, etc. Computing system 200 includes a logic machine 202 and a storage machine 204. Computing system 200 may include a display subsystem 206, input subsystem 208, and communication subsystem 210.

Logic machine 202 may execute machine-readable instructions via one or more physical devices. For example, the logic machine 202 may be configured to execute instructions to perform tasks for a computer program. The logic machine may include one or more processors to execute machine-readable instructions.

Storage machine 204 includes one or more physical devices configured to hold or store instructions executable by the logic machine to implement the method. When such methods and processes are implemented, the state of storage machine 204 may be changed to hold different data. For example, storage machine 204 may include memory devices such as various hard disk drives or CD or DVD devices.

Display subsystem 206 may visually present data stored on storage machine 204. For example, display subsystem 206 may visually present data to form a graphical user interface (GUI). Input subsystem 208 may be configured to connect and receive input from devices such as a mouse, keyboard, or gaming controller. Communication subsystem 210 may be configured to enable system 200 to communicate with other computing devices. Communication subsystem 210 may include wired and/or wireless communication devices to facilitate networked communication.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. A twelve-phase holistic health training system, comprising:
an administrator;
a trainee;
a perspective phase;
an environment phase;
an appreciation phase;
a career phase;
an education phase;
a balance phase;
an exercise phase;
a nutrition phase;
an evolve phase;
a faith phase;
an income phase;
a time phase;
a security phase;
a scoring protocol;
a plurality of point values; and
a progression of scores;
wherein said administrator assesses said trainee based on said scoring protocol to determine a training regimen;
wherein said administrator instructs said trainee in said perspective phase, said environment phase, said appreciation phase, said career phase, said education phase, said balance phase, said exercise phase, said nutrition phase, said evolve phase, said faith phase, said income phase, said time phase, and said security phase over the course of a plurality of training sessions;
wherein said trainee's performance is assessed based on said scoring protocol at the conclusion of each of said plurality of training sessions;
wherein said assessment is tabulated into a plurality of point values; and
wherein said plurality of point values are compiled into a progression of scores.

2. The invention of claim 1, further comprising:
a proprietary software; and
a computing system;
wherein said administrator instructs said trainee in said perspective phase, said environment phase, said appreciation phase, said career phase, said education phase, said balance phase, said exercise phase, said nutrition phase, said evolve phase, said faith phase, said income phase, said time phase, and said security phase via said proprietary software on a visual output of said computing system.

3. A twelve-phase holistic health training method, comprising:
assessing said trainee by said administrator based on said scoring protocol to determine a training regimen;
instructing said trainee by said administrator in said perspective phase, said environment phase, said appreciation phase, said career phase, said education phase, said balance phase, said exercise phase, said nutrition phase, said evolve phase, said faith phase, said income phase, said time phase, and said security phase over the course of a plurality of training sessions;
assessing trainee's performance by said administrator based on said scoring protocol at the conclusion of each of said plurality of training sessions;
tabulating said assessment by said administrator into a plurality of point values; and
compiling said plurality of point values into a progression of scores.
